(19) United States
(12) Patent Application Publication
(10) Pub. No.: US 2007/0105091 A1
(43) Pub. Date: May 10, 2007

(54) Real time assay for rotavirus

(76) Inventor: Charlotte Ip, Blue Bell, PA (US)

Correspondence Address: MERCK AND CO., INC, P O BOX 2000, RAHWAY, NJ, 07065-0907, US

(21) Appl. No.: 10/571,862

(22) PCT Filed: Sep. 15, 2004
(86) PCT No.: PCT/US04/30209

§ 371 Date: Mar. 14, 2006

Related U.S. Application Data
(60) Provisional application No. 60/503,353, filed on Sep. 16, 2003.

(51) Int. Cl. C12Q 1/70; C12Q 1/68; C07H 21/04

(52) U.S. Cl. 435/5; 435/6; 536/24.1

(57) ABSTRACT

A highly sensitive quantitative reverse transcriptase polymerase chain reaction (qRT-PCR) assay with wide linear dynamic range is useful as a high-throughput screening assay for detecting Rotavirus in clinical samples. The primers and probe specifically detect Rotavirus of all serotypes. The assay is particularly useful as a screening assay for determining the cause of diarrhea.

### FIELD OF THE INVENTION

The present invention relates to the field of diagnostics for the detection of rotavirus in clinical samples.

### BACKGROUND OF THE INVENTION

Group A rotaviruses are the most important causative agents of severe acute gastroenteritis in young children worldwide, responsible for 600,000 to 800,000 deaths annually (Ho et al., 1988, JAMA 260:3281-3285; Bern et al., 1996, 1996.Viral infection of the gastrointestinal tract, 2nd ed. New York: Dekker, 1996:1-26; Kapikian and Chanock, 1996, Rotaviruses. In: Fields B N, Knipe D M, Howley P M, et al., editors. Virology, vol. 2. Philadelphia: Lippincott-Raven. 1996:1657-1708). Although the mortality rate is relatively low in developed countries, rotavirus infection is associated with 30 to 60% of hospitalization due to acute gastroenteritis (Brandt et al., 1983, J Clin Microbiol 18:71-78; Glass et al., 1996, J Infect Dis 174 (Suppl) 1:S5-11; Kapildan and Chanock, 1996, Rotaviruses. In: Fields B N, et al., editors. Virology, vol. 2. Philadelphia: Lippincott-Raven. 1996:1657-1708), thereby contributing a significant disease burden to the healthcare system.

Electron microscopy (EM) has been the traditional diagnostic method used since the discovery of the virus in 1973 (Bishop et al., 1973, Lancet 2:1281-1283). However, EM examination of stool samples in routine diagnostic laboratories is limited by the requirement for technical expertise and expensive instrumentation. The usefulness of the technique is further limited because the lower threshold for detection of rotavirus in stool samples by EM is relatively high at 107 viral particles/ml of stool (Madeley et al., 1975, Letter: Viruses in infantile gastroenteritis. Lancet 2:124; McIntosh K. 1996, Diagnostic Virology. In: Fields B N, et al editors. Virology, vol. 1. Lippincott-Raven Publishers, Philadelphia: 1996:401-430 McIntosh, 1996).

Enzyme immunoassays detecting rotavirus antigen have been used as an endpoint assessment in the efficacy trial of rotavirus vaccine (Rennels et al., 1996, Pediatrics 97:7-13; PerezSchael et al., 1997, New Engl J Med 337:1181-1187; Joensuu et al., 1997, Lancet 350:1205-1209). While enzyme immunoassay is 10 to 100 times more sensitive than electron microscopy, the test can be difficult to interpret. False positives can arise from cross-reaction with confounding substances in the sample (Rabenau et al., 1998, Intervirology 41:55-62; Lipson et al., 1990, J Clin Microbiol 28:1132-1134; Dennehy et al., 1988, J Clin Microbiol 26: 1630-1634).

Molecular methods utilizing reverse transcriptase PCR (RT-PCR) have increased the rate of detection of rotaviruses by 15 to 27% in comparison with enzyme immunoassay (Xu et al., 1990, J Virol Methods 27:29-37; Gouvea et al., 1991, J Clin Microbiol 29:519-23; Wilde et al., 1992, J Infect Dis 166:507-511; Pang et al., 1999, J Clin Virol 13:9-16). Real-time PCR represents a technological advance in the molecular diagnostic field that has had many applications. However, data regarding the use of real-time RT-PCR assays for the detection of rotavirus are limited to the SYBR Green I dye method using the real-time cycler Rotorgene 2000 (Schwartz et al. 2002, J. Virol Methods 105:277-285).

A rapid and sensitive assay is needed to provide timely diagnosis of rotavirus for effective clinical management of this disease. The present invention provides a one-step real time quantitative RT-PCR assay for the detection of group A rotavirus using a fluorescent-tagged-label and a closed-tube system. The advantages of the present qRT-PCR assay over previously described assays include greater accuracy, enhanced sensitivity, reliability, fast turn-around-time, high-throughput capability, the minimization of cross-contamination if a close-tube system is used and potential cost savings in labor.

### SUMMARY OF THE INVENTION

An aspect of the invention is a RT-PCR assay for the detection of Rotavirus wherein the forward and reverse primers are derived from a conserved sequence of the NSP gene of Rotavirus.

In particular embodiments of the invention, the forward primer has the sequence of SEQ ID NO: 3 and the reverse primer has the sequence of SEQ ID NO: 4.

In particular embodiments of the invention, the assay is conducted in real time in a closed single tube. In these embodiments, the amplified product is detected using a fluorescently labeled probe. In certain embodiments, the probe is degraded by the DNA polymerase to generate a signal. In other embodiments the probe is a molecular beacon.

In particular embodiments, a control sequence is assayed in parallel with the samples. In these embodiments, the data generated from the control sequence assays is used to derive a standard curve. The assay is then made quantitative by interpreting the sample data in view of the standard curve.

An aspect of this invention is a forward primer having the sequence of SEQ ID NO: 3.

An aspect of this invention is a forward primer having the sequence of SEQ ID NO: 4.

An aspect of this invention is a detection probe having a sequence of SEQ ID NO: 1 that does not overlap with the sequence of SEQ ID NOs:3 or 4. In certain embodiments, the detection probe has the sequence of SEQ ID NO: 2.

### BRIEF DESCRIPTION OF THE DRAWINGS

FIG. 1 Shows data from quantitative RT-PCR of clinical samples.

FIG. 2 Shows a standard curve of the quantitative RT-PCR assay.

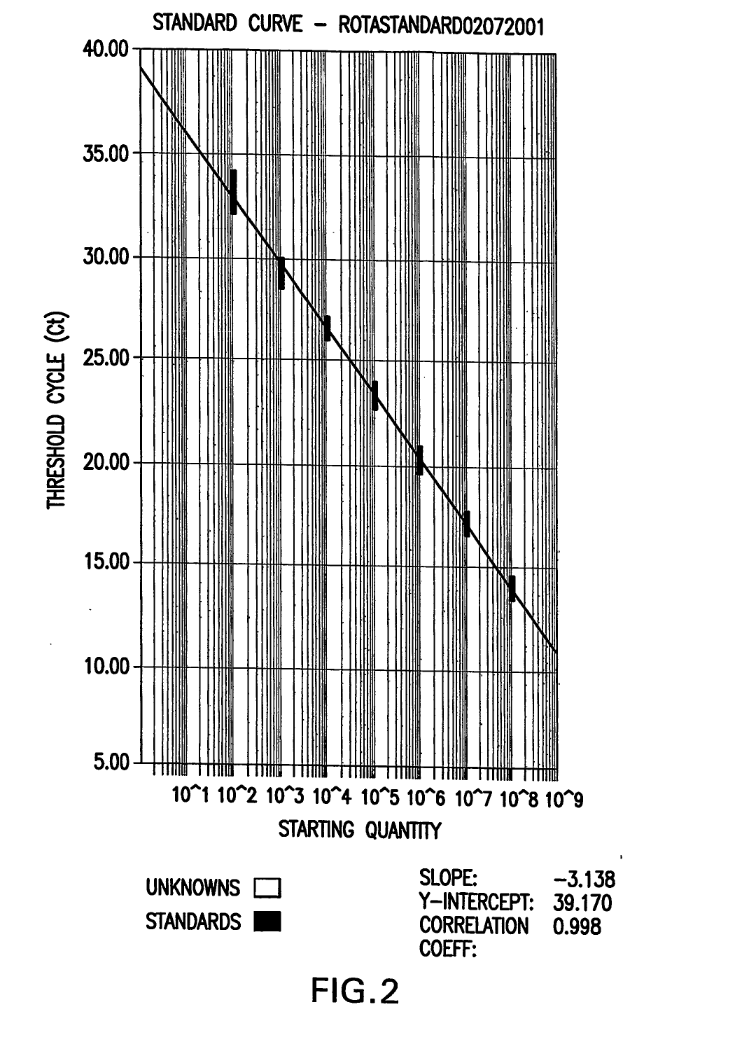

FIG. 3 Shows the sequence of the NSP3 gene (SEQ ID NO: 1) with the PCR primers and probe highlighted in underlined and bold fonts, respectively.

### DETAILED DESCRIPTION OF THE INVENTION

A highly sensitive quantitative reverse transcriptase polymerase chain reaction (qRT-PCR) assay with wide linear dynamic range was developed as a high-throughput screening assay for detecting Rotavirus in stool specimens in children with diarrhea. The primers and probe were designed to specifically detect Rotavirus of all serotypes, based on an 87 bp, 3′UTR conserved region of the NSP3 gene. The assay is useful as a screening assay for determining the cause of diarrhea.

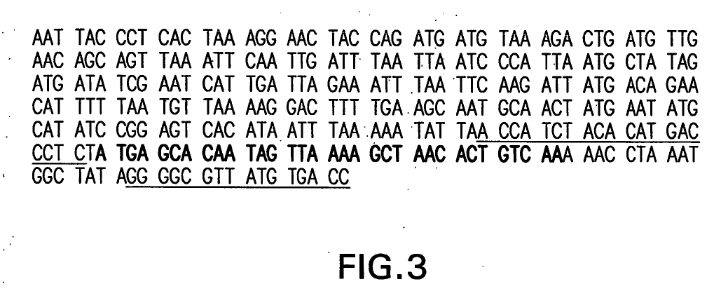

The Rotavirus qRT-PCR assay is based on the detection of a highly conserved NSP3 non-translated region of Rotavirus (See SEQ ID NO: 1, FIG. 3). Rao et al., 1995, Virology, 207, 327-333, reported that a stretch of about 80 nucleotides the 3′ UTR is highly conserved in the NSP3 gene for all strains. The primers are designed to amplify these conserved sequences of NSP3 gene.

A variety of RT-PCR formats are known and used in the art. The primers of the present invention can be employed in many RT-PCR assays known in the art. Preferred embodiments of the invention employ RT-PCR formats that are real-time assays conducted in a closed tube with fluorescent detection.

The qRT-PCR product is detected by means of a fluorogenic probe designed to anneal to a region of SEQ ID NO: 1 between the forward and reverse primers. A 5′ reporter dye and a 3′ quencher dye are attached to the probe. Proximity of the reporter and quencher dyes results in suppression of reporter fluorescence. Upon successful amplification of the target region, the 5′ exonuclease activity of DNA polymerase releases the reporter dye from the hybridized probe, resulting in a fluorescent signal. Numerous reported quencher pairs are known in the art and can be employed in this invention. In preferred embodiments, the reporter/quencher pair is FAM/TAMRA, HEX/TAMRA, TET/TAMRA or 6-FAM/BHQ-1.

In alternative embodiments, the probe can be fashioned as a molecular beacon. Tyagi S and Kramer F R, 1996, Nat Biotechnol 14: 303-308; Tyagi S, et al., 1998, Nat Biotechnol 16: 49-53. When the detection probe is a molecular beacon, the probe sequence of the beacon is chosen from a section of SEQ ID NO: 1 between the forward and reverse primers. Numerous reported quencher pairs are known in the art and can be employed in this invention. In preferred embodiments using molecular beacons the dye pair is 6-FAM/BHQ-1, 6-FAM/Dabcyl, TET/Dabcyl or HEX/Dabcyl.

The fluorescent signal intensity, which is directly proportional to the starting quantity of RNA in a given sample, is monitored by a fluorescence detection system and converted to a value called “cycle threshold” (Ct). The Ct values of plasmid DNA containing the NSP3 gene can be used to generate a standard curve from which quantities of RNA in test samples can be interpolated. Thus, the assay can be used as a qualitative or quantitative measure of viral RNA in the test samples.

The data indicate that using forward and reverse primers of SEQ ID NOs: 3 and 4 and a labeled detection probe of SEQ ID NO: 2, the Rotavirus qRT-PCR assay is 4 logs more sensitive than the conventional RT-PCR and 2 logs more sensitive than the RT-nest-PCR. The reaction time required for the qRT-PCR is about half the time required for the RT-nested-PCR.

The quantitative RT-PCR requires a standard nucleic acid from which to generate a standard curve. A plasmid DNA standard was made containing the conserved region of the NSP3 gene (See FIG. 3). Recognizing that common sources of the PCR contamination are the positive control templates, this plasmid construct may be engineered to contain a unique restriction enzyme site for identifying the false-positive PCR amplified samples. Cleavage of PCR amplified DNA identifies products from the engineered control plasmids. This plasmid is used for generating a standard curve from which quantities of RNA in test samples can be interpolated.

### EXAMPLE 1

General Overview

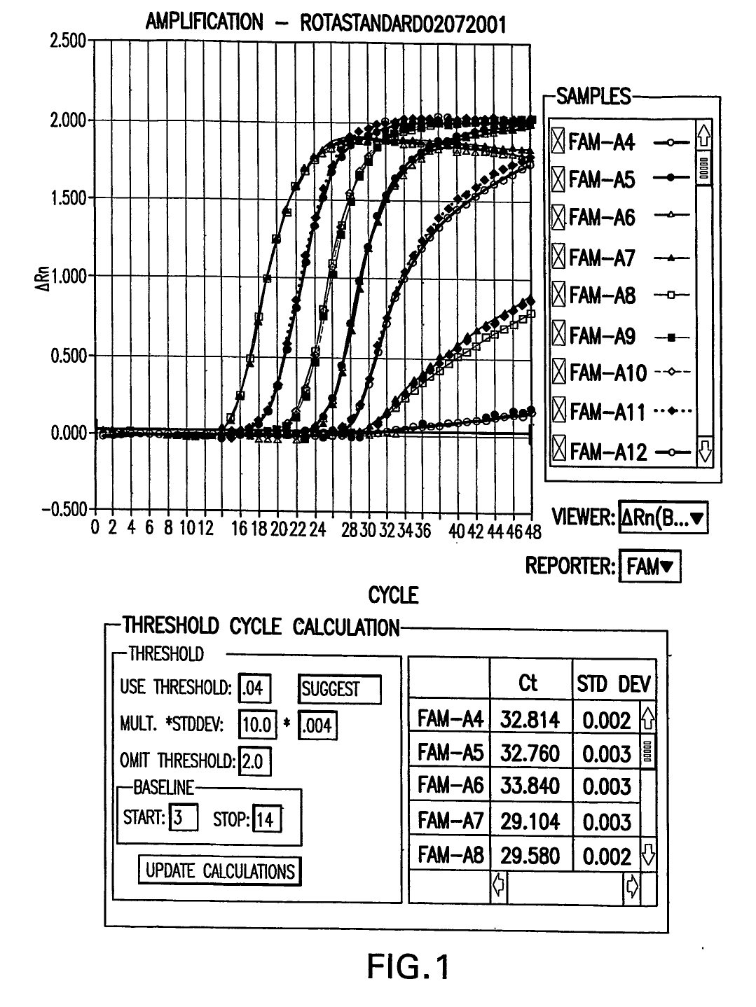

The present quantitative RT-PCR (qRT-PCR) assay is based on the detection of a highly conserved NSP3 non-translated region for the detection of Rotavirus. The DNA sequences of G1 (WI79 & Wa), G2 (DS1, SC2, AE28, AE29, AE 30, AE31, AE32, AE33, AE 34 & AE34), G3 (P, WI78 & R35), G4 (BrB & CC4) and G9 (WI79) have been sequenced and were found to have 100% homology for the primers and probed used in this assay. The assay consisted of an RNA extraction step followed by quantitative RT-PCR using the TAQMAN one step EZ RT-PCR kit (APPLIED BIOSYSTEMS, FOSTER CITY, Calif.) and the plasmid DNA standard containing the Rotavirus NSP3 gene. The RT-PCR amplification plots and the standard curve for 10²-10⁸ are shown on FIGS. 1 & 2.

For rotavirus samples, a QIA AMP Viral RNA kit (QIAGEN, VALENCIA, Calif.) is used to extract RNA prior to RT-PCR. A negative control (PBS) and a positive control (BrB viral stock) are extracted along with samples to check the validity of the assay. Low level contamination (<100 copy) sometimes occurred during extraction of samples with high titers. However, it is possible to minimize the contamination when two physically separate workstations are designated for either reagent or sample preparation. Each workstation is equipped with dedicated equipment and supplies (eg. micro-centrifuge, pipettes, vortex, tips, etc.). In addition, it was found that quality of the extraction of RNA from the sample matrix is critical in obtaining a valid data for the qRT-PCR assay. Cloudy samples or samples containing floating particulate greatly inhibit the efficiency of RNA extraction using the QIA AMP column. For example, in our hands the G2 type samples sometimes gave a lower than expected result because our G2 (SC2) stock was more cloudy in appearance. Table 1 gives the preliminary result for a number of different viral stock samples assayed by classical titering and by qRT-PCR after RNA extraction.

TABLE 1
Viral stock samples assayed by qRT-PCR after RNA extraction

| Sample ID | Copy/mL | Titer, pfu/mL |
|---|---|---|
| G1-Wa | *1.45 × 10⁹ | |
| G1-WI79 | *3.35 × 10⁹ | 1.3 × 10⁷ |
| G2-SC2 | *4.75 × 10⁸ | 1.3 × 10⁶ |
| G2-DS1 | *2.94 × 10⁸ | |
| G3-WI78 | *5.97 × 10⁸ | 6.0 × 10⁵ |
| G3-P | *2.01 × 10⁸ | |
| G4-BrB | *6.14 × 10⁸ | 5.0 × 10⁷ |
| G4-CC4 | *1.65 × 10⁷ | |
| G5-OSU | *3.21 × 10⁷ | |
| G6-WC3 | *7.39 × 10⁶ | 8.0 × 10⁶ |
| G9-WI61 | *2.80 × 10⁹ | |
| P1 | *3.14 × 10⁹ | |
| MLD033-A | *1.26 × 10⁸ | |
| MLD033-B | *1.82 × 10⁸ | |
| MU011-RRV | *4.01 × 10⁸ | |
| MU011-D | *6.72 × 10⁸ | |
| G1-WI79, lot EW115 | **1.3 × 10⁷ | 1.3 × 10⁷ |
| G2-SC2, lot R1240 | **1.3 × 10⁶ | 1.3 × 10⁶ |
| G3-WI78, lot R1243 | **4.5 × 10⁸ | 6.0 × 10⁵ |
| G4-BrB, lot PHB046 | **5.7 × 10⁸ | 5.0 × 10⁷ |
| G6-WC, lot R1182 | **1.9 × 10⁹ | 8.0 × 10⁶ |

*Extracted once
**Extracted twice

Tables 2 & 3 present qRT-PCR data for samples extracted from human stool samples. In table 2, the serotype is given where determined. Some samples used in table 3 were extracted twice with and without additional clarify procedure. It is concluded that sample clarification and RNA extraction of stool suspensions are the critical components of this assay for obtaining valid data. The data also indicated that the qRT-PCR method can be used to detect not only the human rotavirus G types but also the bovine derived reassortant rotaviruses.

TABLE 2
Stool samples assayed by q RT-PCR after RNA extraction

| Sample | Copy/mL |
|---|---|
| G2-STL 1 | *3.29 × 10⁷ |
| G2-STL 2 | *8.38 × 10⁶ |
| Not Determined-STL 4 | *1.81 × 10⁶ |
| G1-STL 5 | *5.37 × 10⁸ |
| G4-STL 6 | *2.52 × 10⁸ |
| G2-STL 7 | *4.94 × 10⁶ |
| G1-STL 8 | *1.62 × 10¹⁰ |
| G2-STL 9 | *2.32 × 10⁹ |
| G2-STL 10 | *9.46 × 10¹¹ |
| G1-STL 11 | *4.42 × 10⁸ |
| G1-STL 12 | *8.80 × 10⁹ |
| G2-STL 13 | *3.67 × 10⁶ |
| G1-STL 14 | *4.42 × 10⁹ |
| G1-STL 15 | *5.34 × 10⁷ |
| G1-STL 16 | *9.92 × 10⁹ |
| G1-STL 17 | *1.92 × 10⁹ |
| G2-STL 18 | *1.93 × 10⁶ |
| G2-STL 19 | *8.49 × 10⁶ |
| G1-STL 20 | *1.39 × 10⁹ |

*Extracted once

TABLE 3
Stool samples assayed by q RT-PCR after RNA extraction

| | | Clarify Suspension |
|---|---|---|
| | | prior to Extraction |
| | Copy #/ | Copy #/ |
| CHMC # | 1 ml suspension | 1 ml suspension |
| 448 | *6.1 × 10⁵ | *2.33 × 10⁶ |
| 449 | *4.63 × 10⁵ | *1.93 × 10⁶ |
| 498 | **1.54 × 10⁸ | |
| 529 | *4.71 × 10⁴ | *1.87 × 10⁵ |
| 535 | *4.13 × 10⁵ | *2.38 × 10⁵ |
| 577 | **1.00 × 10⁹ | |
| 750 | **1.20 × 10⁹ | |
| 798 | **1.76 × 10⁹ | |
| 816 | **9.60 × 10⁸ | |
| 1082 | **2.67 × 10⁸ | |
| 1111 | *2.24 × 10⁶ | *9.09 × 10⁴ |
| 1160 | *7.57 × 10⁵ | *4.39 × 10⁴ |
| 1183 | *2.32 × 10⁶ | *9.04 × 10⁵ |
| 1250 | **1.20 × 10⁷ | |
| 1261 | **1.57 × 10⁸ | |
| 1262 | **2.37 × 10⁸ | |
| 1281 | *6.24 × 10⁶ | *8.51 × 10⁶ |
| 1296 | **6.40 × 10⁸ | |
| 1297 | **4.27 × 10⁸ | |
| 1322 | *2.54 × 10³ | *2.84 × 10⁵ |
| 1347 | *9.37 × 10⁵ | |
| 1368 | *4.07 × 10⁵ | |
| 1393 | *1.14 × 10⁷ | *2.07 × 10⁸ |
| 1394 | **6.40 × 10⁸ | |
| 1430 | **4.80 × 10⁸ | |
| 1434 | **4.53 × 10⁹ | |
| 1435 | **4.53 × 10⁹ | |
| 1512 | **2.67 × 10⁸ | |
| 1513 | **1.39 × 10⁹ | |
| 1561 | **4.27 × 10⁸ | |
| 1562 | **1.87 × 10⁸ | |
| 1597 | **1.81 × 10⁸ | |
| 1598 | **7.20 × 10⁷ | |
| 1601 | **1.11 × 10⁹ | |
| 1602 | **2.00 × 10⁸ | |
| 1616 | **8.00 × 10⁵ | |
| 1617 | **4.10 × 10⁶ | |
| 1618 | **2.93 × 10⁸ | |
| 1619 | **2.03 × 10⁹ | |
| 1624 | **1.73 × 10⁹ | |
| 1625 | **2.93 × 10⁹ | |
| 1632 | **9.60 × 10⁷ | |
| 1633 | *1.97 × 10⁶ | *5.06 × 10⁶ |
| 1636 | *1.44 × 10⁹ | *1.01 × 10⁹ |
| 1637 | **7.47 × 10⁶ | |
| 1774 | *4.41 × 10⁸ | |
| 1775 | *3.78 × 10⁹ | |
| 1792 | **1.15 × 10⁷ | |
| 1793 | **1.84 × 10⁸ | |

**Extracted twice
*Extracted once

### EXAMPLE 2

RNA Extraction

Sample Prepararion and Clarification

Samples are collected in the clinical environment and frozen. After removing samples from frozen storage, immediately add 25× RNASECURE™ Reagent (AMBION, AUSTIN, Tex.) to a 1× final concentration before the samples have thawed. Immediately place the samples in a 65° C. waterbath for 30 mintutes to thaw the samples and activate the RNASECURE™ Reagent. The samples are clarified by centrifuging through 0.45 μm filters, preferably at 6000 rpm in the tabletop centrifuge. This can be done in a 96-well plate format or any 0.45 μm filter individual spin columns, depending on the number of samples.

Viral RNA Isolation

Viral RNA was isolated using the QIAAMP Virus BIOROBOT 9604 Kit (QIAGEN, VALENCIA, Calif, USA) following the manufacturer's instructions. The procedure was performed on the GENISIS RSP 150 Automated Workstation (TECAN, DURHAM, N.C.); however it can also be performed by hand using centrifugation to pull the solutions through the filter.

An aliquot of 200 μl of clarified stool samples are lysed under highly denaturing conditions in the presence of QIAGEN Protease and 2001 μl lysis buffer (Buffer AL) at 70° C. for 10 minutes. The samples are adjusted with 200 μl ethanol, and transferred to a 96-well spin column plate (QIAAMP 96 Plate) where the nucleic acids are absorbed onto the silica-gel membrane as the lysate is drawn through by vacuum pressure. The plate is washed three times using two different wash buffers, which are drawn through by vacuum pressure and centrifugation after the last wash. Viral RNA is eluted in 100 μl room temperature elution buffer (Buffer AVE). Elution volumes can vary, but must be at least 50 μl.

### EXAMPLE 3

Assay

This protocol describes a one-step RT-PCR based fluorogenic 5′ nuclease assay using the APPLIED BIOSYSTEM's (FOSTER CITY, Calif.) TAQMAN EZ RT-PCR kit and the ABI PRISM 7700 sequence detection instrument. The assay targets an 87 bp region of NSP3. This one-step q RT-PCR assay utilizes a plasmid DNA containing 311 bases of the Rotavirus NSP3 gene as a standard.

Materials

1. TaqMan EZ RT-PCR Reagent Kit, (PERKIN ELMER, BOSTON, Mass.), cat.# N808-0236
2. NSP3ROTACONF TaqMan Fluorescent Probe FAM-5′ ATG AGC ACA ATA GTT AAA AGC TAA CAC TGT CAA 3′-TAMRA, (SEQ ID NO: 2) (APPLIED BIOSYSTEMS, FOSTER CITY, Calif.).
3. N5P3ROTA-CON5 Forward Primer, 5′ACC ATC TAC ACA TGA CCC TC3′ (SEQ ID NO: 3)
4. N5P3ROTA-CON3 Reverse Primer, 5′GGT CAC ATA ACG CCC C3′ (SEQ ID NO: 4)
5. Rota-NSP3 plasmid DNA standard, 1.2 mg/ml
6. Molecular Biology Grade Water (DNase, RNase, and Protease free) 4 Liter, 5′-3′, Inc., cat. 5302-336550
7. Salmon Sperm DNA, (SIGMA, ST LOUIS, Mo.), cat. # D7656, 9.8 mg/ml
8. 2% Solution Gelatin, (SIGMA ALDRICH, ST LOUIS, Mo.), cat. # G1393
9. Polyoxyethylene Sorbitan Monolaurate Molecular Biology Grade, (SIGMA ALDRICH, ST LOUIS, Mo.), cat. # P9416
10. RNA and DNA Zap Solution 1 and 2, (AMBION, AUSTIN, Tex.), cat. # 9890
11. Extracted RNA samples including positive (BrB Rotavius stock) and negative (PBS) samples.
   Equipment

We have used MICRO AMP optical tubes, caps and base (APPLIED BIOSYSTEMS, FOSTER CITY, Calif.) but equivalent equipment can be used as desired. Our preferred detector system is the ABI Prism 7700 Sequence detector (APPLIED BIOSYSTEMS, FOSTER CITY, Calif.) with a computer for analysis.

Preparation for PCR

Minimizing the Risk of Contamination Preparation for PCR Preparation for PCR

In order to minimize the risk of contamination of the samples, the assay. was performed in three separate rooms. Gloves were changed between each step of the procedure to prevent cross contamination. Master mixes for PCR steps are prepared in a hood in the first “clean” room where no test samples or positive controls are kept. The second room is used for the dilution and loading of any unknown samples with a separate set of pipettes. The third room is used for dilution and loading of positive controls with a separate set of pipettes.

Preparation of 5.0 μg/ml Salmon Sperm DNA

Place 5 μl of 9.8 mg/ml salmon sperm DNA (stock solution) into a 15.0-ml screw cap tube. Add 9.795 ml molecular biology grade water and vortex to mix well. This is the 5.0 μg/ml salmon sperm DNA solution to be used as a carrier for dilution of standards. This solution can be stored for period of 3 months at 4° C.

Preparation of PCR 10× ROX Stabilizer (0.5% gelatin, 0.1% Tween 20)

Weigh˜45 mg of Tween 20 by pipetting˜50 μl Tween 20 into a 2-ml microcentrifuge tube. Add 950 μl of molecular biology grade water, mix and transfer to a 50-ml graduated conical tube. Add 11.25 ml of 2% gelatin. Add 32.75 ml of molecular biology grade water. The final volume should be 45 ml.

Preparation of Primers and Probe (Stored at −20 Celsius)

The reverse and forward primers and TAQMAN fluorescent probe may be synthesized by methods well known in the art and commercially available. Prior to use, the primers are dissolved in molecular biology grade water at a concentration of 10 uM. The solutions are stored at −20 Celsius, and are good for period of about 1 year.

Master Mix Preparation for PCR Reaction

The following reagents are placed into a sterile RNase ZAP treated 15-ml tube. The final volume in the tube is 4500 μl The final concentration refers to the concentration in the 45 μl PCR reaction volume consisting of 5 μl sample and 45 μl master mix. This master mix volume is for 100 reactions; however, the amount can be varied, (i.e. halved, doubled, tripled, etc.) depending on the number of PCR reactions to be run. The following mixture can be stored at 4° C. for period of 1 month.

Preparation of qRT-PCR Master Mix

| | Volume for 100 | Final | |
|---|---|---|---|
| Reagent | reactions (ul) | Concentration | |
| 5x TAQMAN EZ Buffer | 1000 | 1x | |
| Manganese acetate (25 mM) | 1000 | 5 | mM |
| dATP (10 mM) | 150 | 300 | uM |
| dCTP (10 mM) | 150 | 300 | uM |
| dGTP (10 mM) | 150 | 300 | uM |
| dUTP (20 mM) | 150 | 600 | uM |
| rTH DNA Polymerase (2.5 U/ul) | 200 | 0.1 | U/ul |
| AMPERASE UNG (1 U/ul) | 50 | 0.01 | U/ul |
| N5P3ROTA-CON5 primer (10 uM) | 250 | 500 | nM |
| N5P3ROTA-CON3 primer (10 uM) | 250 | 500 | nM |
| NSP3ROTACONF probe (10 uM) | 100 | 200 | nM |
| Rox Stabilizer | 500 | | |
| Molecular Biology Grade water | 550 | | |
| volume (ul) | | | |

Preparation of Standard

The Control standard dilution series is carried out in 5 μg/ml of salmon sperm DNA in sterile 2 ml screw cap tubes as follow:

| | | | | Salmon | | | |
|---|---|---|---|---|---|---|---|
| | | | Volume | Sperm | Final | | Copy/ |
| Tube # | Rota-NSP3 | | (ul) | DNA (ul) | concentration | | reaction |
| 1 | 1.2 | mg/ml | 1 | 999 | 1.2 | ug/ml | |
| 2 | 1200 | ng/ml | 234 | 66 | 936 | ng/ml | |
| 4 | 936 | ng/ml | 20 | 180 | 93.6 | ng/ml | 10⁸ |
| 5 | 936 | ng/ml | 2 | 198 | 9.36 | ng/ml | 10⁷ |
| 6 | 9.36 | ng/ml | 20 | 180 | 936 | pg/ml | 10⁶ |
| 7 | 9.36 | ng/ml | 2 | 198 | 93.6 | pg/ml | 10⁵ |
| 8 | 93.6 | pg/ml | 20 | 180 | 9.36 | pg/ml | 10⁴ |
| 9 | 93.6 | pg/ml | 2 | 198 | 936 | fg/ml | 10³ |
| 10 | 936 | fg/ml | 20 | 180 | 93.6 | fg/ml | 10² |

An example of the calculation for copy number for 5 μl of the 936 fg/ml sample solution assayed in a PCR reaction is shown below.
936 fg/ml DNA and 0.005 ml volume used for each PCR reaction=4.68 fg
Rota-NSP3 plasmid**=4268 bp and 1 bp=660 g/bp
Avogadro number=6.023×1023 copy/mole
660 g/bp×4268 bp/mole=2.816×106 g /mole
(6.023×1023 copy/mole*4.68 fg)/(2.816×106 g/mole×1015 fg/g)=1000 copy

**Rota-NSP3 plasmid: 3957 bp (vector)+311 bp (RotaNSP3 gene insert)=4268 bp

Setting up PCR Reactions

The reactions were prepared in 96 well plates. If the plates come with retainers, they can be removed and discarded. The PCR plate is placed on MICROAMP base. Optical tubes are labeled to match the numbering on the PCR plate, and the tubes are placed on the PCR plate accordingly. If desired, programmable pipettes can be employed to dispense reagents.

Master mix is dispensed, 45 ul, into each tube. The negative control samples having salmon sperm DNA are prepared and capped with a capping tool. Samples are then dispensed in triplicate and capped immediately. Positive controls are prepared by adding 5 μl of salmon sperm DNA (5 μg/mL), plasmid standards, PBS and wild type BrB rotavirus stock respectively, as desired, in triplicate in order of low concentration range to high concentration range. The PCR tubes are capped immediately after each triplicate was added.

The thermal cycler is programmed to cycle as follows: 50.0° C. for 2 minutes; 60.0° C. for 30 minutes; 95.0° C. for 5 minutes; 94.0° C. for 0.20 minutes; 51.0° C. for 1 minute; for 48 cycles and finally 25.0° C. for 2 minutes. During the PCR cycles the fluorescence in each tube is recorded and stored.

Results

When the RT-PCR run is finished, the recorded data is analyzed. The data in the FAM dye layer is noted. To obtain the standard curve calibration plot for the FAM layer, one views the data of the standard plate(s). To analyze the experimental data for the FAM layer, one refers to the samples.

What is claimed:

1. A real time qRT-PCR assay for the detection of Rotavirus wherein the forward primer is 5′ACC ATC TAC ACA TGA CCC TC3′ (SEQ ID NO: 3), the reverse primer is 5′GGT CAC ATA ACG CCC C3′ (SEQ ID NO: 4) and the confirmation primer is selected from a sequence of SEQ ID NO: 1 and does not overlap with SEQ ID NO: 3 or SEQ ID NO: 4.

2. The assay of claim 1 wherein the confirmation primer is 5′ ATG AGC ACA ATA GTT AAA AGC TAA CAC TGT CAA 3′ (SEQ ID NO: 2).

3. The assay of claim 2 wherein the 5′ end of the confirmation primer is labeled with FAM and the 3′ end of the confirmation primer is labeled with TAMRA.

4. The assay of claim 1 wherein the confirmation primer is a molecular beacon.

5. The assay of claim 4 wherein the molecular beacon contains at least 25 nucleotides of SEQ ID NO: 2.

6. The assay of claim 1 wherein the assay is quantitative of the amount of Rotavirus detected.

7. A primer having the sequence of SEQ ID NO: 3.

8. A primer having the sequence of SEQ ID NO: 4.

9. A primer having the sequence of SEQ ID NO: 2.

10. The primer of claim 9 further comprising a FAM label on the 5′ end of the primer and a TAMRA label on the 3′ end of the primer.


SEQUENCE LISTING (4 sequences)

SEQ ID NO: 1 - DNA, 311 bp, rotavirus
aattaccctcactaaaggaactaccagatgatgtaaagactgatgttgaacagcagttaaattcaattgatttaattaatcccattaatgctatagatgatatcgaatcattgattagaaatttaattcaagattatgacagaacatttttaatgttaaaaggacttttgaagcaatgcaactatgaatatgcatatccggagtcacataatttaaaaatattaaccatctacacatgaccctctatgagcacaatagttaaaagctaacactgtcaaaaacctaaatggctataggggcgttatgtgacc

SEQ ID NO: 2 - DNA, 33 bp, rotavirus
atgagcacaatagttaaaagctaacactgtcaa

SEQ ID NO: 3 - DNA, 20 bp, rotavius
accatctacacatgaccctc

SEQ ID NO: 4 - DNA, 16 bp, rotavirus
ggtcacataacgcccc